(19) United States
(12) Patent Application Publication
(10) Pub. No.: US 2020/0171087 A1
(43) Pub. Date: Jun. 4, 2020

(54) METHODS FOR TREATING MERKEL CELL CARCINOMA (MCC) USING NK-92 CELLS

(71) Applicant: NANTKWEST, INC., San Diego, CA (US)

(72) Inventors: Hans Klingemann, Boston, MA (US); Tien Lee, San Diego, CA (US); Laurent Boissel, Brookline, MA (US)

(73) Assignee: NANTKWEST, INC., San Diego, CA (US)

(21) Appl. No.: 16/620,855

(22) PCT Filed: Jun. 19, 2018
(86) PCT No.: PCT/US2018/038308

§ 371 Date: Dec. 9, 2019

Related U.S. Application Data
(60) Provisional application No. 62/522,335, filed on Jun. 20, 2017.

(51) Int. Cl. A61K 35/17; A61P 35/04; A61K 9/00

(52) U.S. Cl. CPC A61K 35/17; A61K 9/0029; A61P 35/04

(57) ABSTRACT

Provided herein are methods of treating merkel cell carcinoma. The methods include selecting a subject having merkel cell carcinoma and administering to the subject a therapeutically effective amount of NK-92 cells, wherein administration treats the merkel cell carcinoma in the subject.

### RELATED APPLICATION

This application claims the benefit of U.S. Provisional Application No.: 62/522,335, filed on Jun. 20, 2017. This provisional application is incorporated by reference herein in its entirety for all purposes.

### BACKGROUND

MCC is a rare but increasingly common, aggressive skin cancer (0.79 cases per 100,000 person-years in the United States (Fitzgerald, et al., Am. Surg. 81:802-6 (2015)), and incidence rates of the disease have tripled over the past 15 years (Banks, et al., J Oncol Pract. 12:637-46 (2016)). MCC was first proposed to arise from Merkel cells, which are slowly adapting mechanoreceptors of the skin; however, the source of tumor cells remains poorly understood, and pluripotent stem cells and epidermal keratinocyte-like cells may give rise to cancer cells (Tilling and Moll, J Skin Cancer. 2012:680410 (2012)). MCC is more common in Caucasians, individuals >65 years old, men, and patients with acquired (e.g., HIV infection) or iatrogenic immune suppression (e.g., due to treatment of autoimmune diseases) (Becker, Ann Oncol. 21 Suppl 7:vii81-5 (2010)). Ultraviolet exposure is an independent risk factor for the disease and may contribute to the rising incidence of MCC.

MCC that is confined to the skin has a good prognosis and can often be cured by surgery alone. The five-year overall survival (OS) rate for patients presenting with local disease is 66% for tumors <2 cm and 51% for tumors >2 cm. Metastatic MCC has a much poorer prognosis, with five-year OS of 39% for patients with regional lymph node involvement and 18% for those with metastases to distant organs (Lemos, et al., J Am Acad Dermatol. 63:751-61 (2010)). Disease stage, location (perineum and lower extremities), male gender, advanced age (>60 years old), immunosuppression, comorbid factors, high mitotic rate, and angiolymphatic invasion are associated with poor prognosis (Becker, Ann Oncol. 21 Suppl 7:vii81-5 (2010); and Miller, et al., Curr Treat Options Oncol. 14:249-63 (2013)).

Surgical resection is the cornerstone of therapy for MCC, with the goal of establishing clear surgical margins by wide local excision. Adjuvant radiation therapy to the primary tumor bed in patients with stage I/II MCC has been shown to improve OS (Bhatia, et al., J Natl Cancer Inst. 108 (2016)); the same study reported that neither systemic chemotherapy nor radiation therapy in patients with stage III disease improves OS (Bhatia, et al., J Natl Cancer Inst. 108 (2016)), although other studies suggest chemotherapy may increase survival in patients with advanced MCC (Poulsen, J Clin Oncol. 21:4371-6 (2003)).

Cytotoxic chemotherapy is often used to treat metastatic MCC. A minority of patients treated with chemotherapy respond well to treatment, but responses are usually transient and rarely lead to significant increases in survival time (Iyer, et al., Cancer Med. (2016)). Adjuvant treatment with etoposide and carboplatin has not been associated with OS benefit for patients with advanced locoregional disease (Poulsen, et al., Int J Radiat Oncol Biol Phys. 64:114-9 (2006)). Some studies have demonstrated high objective antitumor responses (>50%) using cytotoxic chemotherapy (etoposide-carboplatin and cyclophosphamide-doxorubicin-vincristine-prednisone have been the most frequently used) in patients with metastatic MCC (Voog, Cancer. 85:2589-95 (1999)); however, these responses are rarely durable, with median OS of 9 months. Moreover, high rates of chemotoxic death were associated with first-line treatments. At present, limited data exists to guide treatment decisions regarding chemotherapy and radiotherapy, and often decisions are made based on comorbidities and consideration of adverse events (Lebbe, et al., Eur J Cancer. 51:2395-403 (2015)).

A limited number of studies have investigated the efficacy of targeted therapies against advanced MCC. In phase 2 clinical studies, treatment with the tyrosine kinase inhibitor, imatinib (23 patients) resulted in one partial response (Samlowski, et al., Am J Clin Oncol., 33:495-9 (2010); and Shah, et al., Am J Clin Oncol. 32:174-9 (2009)), and treatment with the Bcl-2 antisense oligonucleotide, G3139 (12 patients), did not yield any objective responses (Samlowski, et al., Am J Clin Oncol., 33:495-9 (2010); and Shah, et al., Am J Clin Oncol. 32:174-9 (2009)).

In a phase 1 study, pembrolizumab, an anti-PD1 therapeutic antibody, was assessed in 30 patients with advanced solid tumors. The single patient enrolled in the study who had MCC had a complete response that was ongoing at the time of publication (100+weeks) (Patnaik, et al., Clin Cancer Res. 21:4286-93 (2015)).

In a recent phase 2 clinical study focused exclusively on MCC, 25 patients with advanced MCC received at least one dose of pembrolizumab and were evaluated for treatment response (Nghiem, N Engl J Med. 374:2542-52 (2016)). All patients had distant metastatic or local recurrent MCC that was not amenable to definitive surgery or radiotherapy. Pembrolizumab was administered IV at 2 mg/kg every three weeks, and treatment was continued for a maximum of 2 years, or until a complete response, progressive disease, or dose-limiting toxic effects occurred. The objective response rate in this study was 56%, with 4 patients exhibiting a complete response and 10 a partial response. Among patients demonstrating an objective response, response durations ranged from a minimum of at least to 2.2 months to a maximum duration of at least 9.7 months. Responses were observed in both MCV-positive (10 of 16 patients) and MCV-negative tumors (4 of 9 patients). Grade 3 or 4 treatment-related adverse events were observed in 4 patients, with the most serious AEs including myocarditis and elevated levels of aspartate and alanine aminotransferase.

Current treatments for MCC are ineffective, partially effective or result in adverse side effects. Therefore, additional treatments for MCC are needed.

### BRIEF SUMMARY

Provided herein are methods of treating merkel cell carcinoma. The methods include selecting a subject having merkel cell carcinoma and administering to the subject a therapeutically effective amount of NK-92 cells, wherein administration treats the merkel cell carcinoma in the subject.

### BRIEF DESCRIPTION OF THE DRAWINGS

FIG. 1 is a graph showing cytotoxic effects of NK-92 cells against merkel cell carcinoma cell lines at 4 hours.

FIG. 2 is a graph showing cytotoxic effects of NK-92 cells against merkel cell carcinoma cell lines at 24 hours.

### DETAILED DESCRIPTION

Because of the viral origin for this cancer, immunotherapies may be a promising avenue for research to treat merkel cell carcinoma. Provided herein are methods of treating merkel cell carcinoma. The methods include selecting a subject having merkel cell carcinoma and administering to the subject a therapeutically effective amount of NK-92 cells, wherein administration treats the merkel cell carcinoma in the subject.

The NK-92 cell line is a human, IL-2-dependent NK cell line that was established from the peripheral blood mononuclear cells (PBMCs) of a 50-year-old male diagnosed with non-Hodgkin lymphoma (Gong, et al., Leukemia. 8:652-8 (1994)). NK-92 cells are characterized by the expression of CD56^{bright} and CD2, in the absence of CD3, CD8, and CD16. A CD56^{bright}/CD16^{neg}/low phenotype is typical for a minor subset of NK cells in peripheral blood, which have immunomodulatory functions as cytokine producers. Unlike normal NK cells, NK-92 lacks expression of most killer cell inhibitor receptors (KIRs) (Maki, et al., J Hematother Stem Cell Res. 10:369-83 (2001)). Only KIR2DL4, a KIR receptor with activating function and inhibitory potential that is expressed by all NK cells, was detected on the surface of NK-92. KIR2DL4 is considered to mediate inhibitory effects through binding to the HLA allele G (Suck, Cancer Immunol. Immunother. 65(4):485-92 (2015)). The predominant pathway of cytotoxic killing of NK-92 cells is through the perforin/esterase pathway; NK-92 expresses high levels of perforin and granzyme B (Maki, et al., J Hematother Stem Cell Res. 10:369-83 (2001)).

NK-92 cells have a very broad cytotoxic range and are active against cell lines derived from hematologic malignancies and solid tumors (Klingemann, Blood, 87(11):4913-4 (1996); Swift, Haematologica. 97(7):1020-8 (2012); Yan, et al., Clin Cancer Res. 4:2859-68 (1998)). Safety assessments in severe combined immunodeficiency (SCID) mice showed no NK-92 treatment-related effects, such as acute toxicity or long-term carcinogenicity (Tam, et al., J Hematother. 8:281-90 (1999), Yan, et al., Clin Cancer Res. 4:2859-68 (1998)). Administration of NK-92 cells to mice challenged with human leukemia cells or mouse models of human melanoma resulted in improved survival and suppression of tumor growth, including complete remissions in some mouse tumors (Tam, et al., J Hematother. 8:281-90 (1999), Yan, et al., Clin Cancer Res. 4:2859-68 (1998)). Phase I clinical trials have confirmed its safety profile. Characterization of the NK-92 cell line is disclosed in WO 1998/49268 and U.S. Patent Application Publication No. 2002-0068044, which are incorporated by reference herein in their entireties.

Provided herein are methods of treating merkel cell carcinoma in a subject. The methods include selecting a subject having merkel cell carcinoma and administering to the subject a therapeutically effective amount of NK-92 cells, wherein administration treats the merkel cell carcinoma in the subject. Optionally, the subject has previously received radiation therapy, surgery, chemotherapy, anti-PD-1 therapy or any combination thereof. Optionally, the merkel cell carcinoma is metastatic. Optionally, the merkel cell carcinoma is caused by the merkel cell polyomavirus. Optionally, the merkel cell carcinoma is not caused by the merkel cell polyomavirus. Optionally, the merkel cell carcinoma in the subject is resistant to chemotherapy.

As used herein, the term “cancer” refers to all types of cancer, neoplasm, or malignant tumors found in mammals, including leukemia, carcinomas and sarcomas.

Exemplary cancers include cancer of the brain, breast, cervix, colon, head & neck, liver, kidney, lung, non-small cell lung, melanoma, mesothelioma, ovary, sarcoma, stomach, uterus and Medulloblastoma. Additional examples include, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas, and prostate cancer.

As used herein, the term “merkel cell carcinoma” refers to a neuroendocrine carcinoma of the skin. It is also known as cutaneous APUDoma, primary small cell carcinoma of the skin, and trabecular carcinoma of the skin. The term “merkel cell carcinoma” includes merkel cell carcinomas caused by the merkel cell polyomavirus as well as those arising from other origins.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The terms subject, patient, individual, etc. are not intended to be limiting and can be generally interchanged. That is, an individual described as a patient does not necessarily have a given disease, but may be merely seeking medical advice. As used throughout, a subject can be a vertebrate, more specifically a mammal (e.g., a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient, individual and subject may be used interchangeably and these terms are not intended to be limiting. That is, an individual described as a patient does not necessarily have a given disease, but may be merely seeking medical advice. The terms patient or subject include human and veterinary subjects.

“Administration” or “administering,” as used herein, refers to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional or intracranial injection), transdermal, topical, buccal, rectal, vaginal, nasal, ophthalmic, via inhalation, and implants. Optionally, the NK-92 cells are administered parenterally. Optionally, the NK-92 cells are administered intravenously. Optionally, the NK-92 cells are administered peritumorally.

The NK-92 cells may be administered to the subject by a variety of routes. For example, the NK-92 cells can be administered to the subject by infusion (e.g., intravenous infusion) over a period of time. Typically, for a single dose of NK-92 cells, the period of time is between 5 and 130 minutes. Optionally, the period of time is between 90 and 120 minutes. Optionally, the period of time is between 15 to 30 minutes.

The NK-92 cells, and optionally other anti-cancer agents can be administered once to a patient with cancer can be administered multiple times, e.g., once every 1, 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10 or more weeks during therapy, or any ranges between any two of the numbers, end points inclusive. Thus, for example, NK-92 cells can be administered to the subject once daily for 1, 2, 3, 4, 5, 6, 7, 8, 9, 10, 11, 12, 13, 14, 15, 16, 17, 18, 19, 20 or more days. Optionally, the NK-92 cells are administered in a cycle of once daily for two days. The cycle is then followed by one or more hours, days, or weeks of no treatment with NK-92 cells. As used herein, the term “cycle” refers to a treatment that is repeated on a regular schedule with periods of rest (e.g., no treatment or treatment with other agents) in between. For example, treatment given for one week followed by two weeks of rest is one treatment cycle. Such cycles of treatment can be repeated one or more times. Thus, the NK-92 cells can be administered in 1, 2, 3, 4, 5, 6, 7, 8, 9, 10, 11, 12, 13, 14, 15, 16, 17, 18, 19, 20 or more cycles.

NK-92 cells can be administered to a subject by absolute numbers of cells, e.g., said subject can be administered from about 1000 cells/injection to up to about 10 billion cells/injection, such as at about, at least about, or at most about, 1×10¹⁰, 1×10⁹, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) NK-92 cells per injection, or any ranges between any two of the numbers, end points inclusive. Optionally, from 1×10⁸ to 1×10¹⁰ cells are administered to the subject. Optionally, the cells are administered one or more times weekly for one or more weeks. Optionally, the cells are administered once or twice weekly for 1, 2, 3, 4, 5, 6, 7, 8, 9, 10 or more weeks.

Optionally, subject are administered from about 1000 cells/injection/m² to up to about 10 billion cells/injection/m², such as at about, at least about, or at most about, 1×10¹⁰/m², 1×10⁹/m², 1×10⁸/m², 1×10⁷/m², 5×10⁷/m², 1×10⁶/m², 5×10⁶/m², 1×10⁵/m², 5×10⁵/m², 1×10⁴/m², 5×10⁴/m², 1×10³/m², 5×10³/m² (and so forth) NK-92 cells per injection, or any ranges between any two of the numbers, end points inclusive. Optionally, from 1×10³ to 1×10¹⁰, per m² of the NK-92 cells are administered to the subject. Optionally, 2×10⁹ per m², of the NK-92 cells are administered to the subject.

Optionally, NK-92 cells can be administered to such individual by relative numbers of cells, e.g., said individual can be administered about 1000 cells to up to about 10 billion cells per kilogram of the individual, such as at about, at least about, or at most about, 1×10¹⁰, 1×10⁹, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) NK-92 cells per kilogram of the individual, or any ranges between any two of the numbers, end points inclusive.

Optionally, the total dose may calculated by m² of body surface area, including about 1×10¹¹, 1×10¹⁰, 1×10⁹, 1×10⁸, 1×10⁷, per m², or any ranges between any two of the numbers, end points inclusive. Optionally, between about 1 billion and about 3 billion NK-92 cells are administered to a patient. Optionally, the amount of NK-92 cells injected per dose may calculated by m2 of body surface area, including 1×10¹¹, 1×10¹⁰, 1×10⁹, 1×10⁸, 1×10⁷, 1×10⁶, 1×10⁵, 1×10⁴, 1×10³, per m².

Optionally, NK-92 cells are administered in a composition comprising NK-92 cells and a medium, such as human serum or an equivalent thereof. Optionally, the medium comprises human serum albumin. Optionally, the medium comprises human plasma. Optionally, the medium comprises about 1% to about 15% human serum or human serum equivalent. Optionally, the medium comprises about 1% to about 10% human serum or human serum equivalent. Optionally, the medium comprises about 1% to about 5% human serum or human serum equivalent. Optionally, the medium comprises about 2.5% human serum or human serum equivalent. Optionally, the serum is human AB serum. Optionally, a serum substitute that is acceptable for use in human therapeutics is used instead of human serum. Such serum substitutes may be known in the art. Optionally, NK-92 cells are administered in a composition comprising NK-92 cells and an isotonic liquid solution that supports cell viability. Optionally, NK-92 cells are administered in a composition that has been reconstituted from a cryopreserved sample.

According to the methods provided herein, the subject is administered an effective amount of one or more of the agents provided herein. The terms effective amount and effective dosage are used interchangeably. The term effective amount is defined as any amount necessary to produce a desired physiologic response (e.g., reduction of inflammation). Effective amounts and schedules for administering the agent may be determined empirically by one skilled in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 22nd Edition, Gennaro, Editor (2012), and Pickar, Dosage Calculations (1999)).

Pharmaceutically acceptable compositions can include a variety of carriers and excipients. A variety of aqueous carriers can be used, e.g., buffered saline and the like. These solutions are sterile and generally free of undesirable matter. Suitable carriers and their formulations are described in Remington: The Science and Practice of Pharmacy, 22nd Edition, Loyd V. Allen et al., editors, Pharmaceutical Press (2012). By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient and to minimize adverse side effects in the subject. As used herein, the term pharmaceutically acceptable is used synonymously with physiologically acceptable and pharmacologically acceptable. A pharmaceutical composition will generally comprise agents for buffering and preservation in storage and can include buffers and carriers for appropriate delivery, depending on the route of administration.

The compositions may contain acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of cells in these formulations and/or other agents can vary and will be selected primarily based on fluid volumes, viscosities, body weight and the like in accordance with the particular mode of administration selected and the subject's needs.

Optionally, the NK-92 cells are administered to the subject in conjunction with one or more other treatments for the cancer being treated. Without being bound by theory, it is believed that co-treatment of a subject with NK-92 cells and another therapy for the cancer will allow the NK-92 cells and the alternative therapy to give the endogenous immune system a chance to clear the cancer that heretofore had overwhelmed such endogenous action. Optionally, two or more other treatments for the cancer being treated includes, for example, an antibody, radiation, chemotherapeutic, stem cell transplantation, or hormone therapy. Optionally, an antibody is administered to the patient in conjunction with the NK-92 cells. Optionally, the NK-92 cells and an antibody are administered to the subject together, e.g., in the same formulation; separately, e.g., in separate formulations, concurrently; or can be administered separately, e.g., on different dosing schedules or at different times of the day. When administered separately, the antibody can be administered in any suitable route, such as intravenous or oral administration.

Optionally, antibodies may be used to target cancerous cells or cells that express cancer-associated markers. A number of antibodies have been approved for the treatment of cancer, alone.

The provided methods may be further combined with other tumor therapies such as radiotherapy, surgery, hormone therapy and/or immunotherapy. Thus, the provided methods can further include administering one or more additional therapeutic agents to the subject. Suitable additional therapeutic agents include, but are not limited to, analgesics, anesthetics, analeptics, corticosteroids, anticholinergic agents, anticholinesterases, anticonvulsants, antineoplastic agents, allosteric inhibitors, anabolic steroids, antirheumatic agents, psychotherapeutic agents, neural blocking agents, anti-inflammatory agents, antihelmintics, antibiotics, anticoagulants, antifungals, antihistamines, antimuscarinic agents, antimycobacterial agents, antiprotozoal agents, antiviral agents, dopaminergics, hematological agents, immunological agents, muscarinics, protease inhibitors, vitamins, growth factors, and hormones. The choice of agent and dosage can be determined readily by one of skill in the art based on the given disease being treated. Optionally, the additional therapeutic agent is octreotide acetate, interferon, pembrolizumab, glucopyranosyl lipid A, carboplatin, etoposide, or any combination thereof.

Optionally, the additional therapeutic agent is a chemotherapeutic agent. A chemotherapeutic treatment regimen can include administration to a subject of one chemotherapeutic agent or a combination of chemotherapeutic agents. Chemotherapeutic agents include, but are not limited to, alkylating agents, anthracyclines, taxanes, epothilones, histone deacetylase inhibitors, inhibitors of Topoisomerase I, inhibitors of Topoisomerase II, kinase inhibitors, monoclonal antibodies, nucleotide analogs and precursor analogs, peptide antibiotics, platinum-based compounds, retinoids, and vinca alkaloids and derivatives. Optionally, the chemotherapeutic agent is carboplatin.

Combinations of agents or compositions can be administered either concomitantly (e.g., as a mixture), separately but simultaneously (e.g., via separate intravenous lines) or sequentially (e.g., one agent is administered first followed by administration of the second agent). Thus, the term combination is used to refer to concomitant, simultaneous, or sequential administration of two or more agents or compositions. The course of treatment is best determined on an individual basis depending on the particular characteristics of the subject and the type of treatment selected. The treatment, such as those disclosed herein, can be administered to the subject on a daily, twice daily, bi-weekly, monthly, or any applicable basis that is therapeutically effective. The treatment can be administered alone or in combination with any other treatment disclosed herein or known in the art. The additional treatment can be administered simultaneously with the first treatment, at a different time, or on an entirely different therapeutic schedule (e.g., the first treatment can be daily, while the additional treatment is weekly).

Also disclosed are kits comprising the provided NK-92 cells for treating merkel cell carcinoma. Optionally, the kit may contain additional compounds such as therapeutically active compounds or drugs that are to be administered before, at the same time, or after administration of the NK-92 cells. Examples of such compounds include vitamins, minerals, fludrocortisone, ibuprofen, lidocaine, quinidine, chemotherapeutic agents, and the like. Optionally, the kit includes an injection device. As used herein, “injection device” refers to a device that is designed for carrying out injections, an injection including the steps of temporarily fluidically coupling the injection device to a person's tissue, typically the subcutaneous tissue. An injection further includes administering an amount of an agent into the tissue and decoupling or removing the injection device from the tissue. In some embodiments, an injection device can be an intravenous device or IV device, which is a type of injection device used when the target tissue is the blood within the circulatory system, e.g., the blood in a vein. A common, but non-limiting example of an injection device is a needle and syringe.

Optionally, instructions for use of the kits will include directions to use the kit components in the treatment of a cancer. The instructions may further contain information regarding how to prepare (e.g., dilute or reconstitute, in the case of freeze-dried protein) the antibody and the NK-92 cells (e.g., thawing and/or culturing). The instructions may further include guidance regarding the dosage and frequency of administration.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed while, specific references to each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

The examples below are intended to further illustrate certain aspects of the methods and compositions described herein, and are not intended to limit the scope of the claims.

### EXAMPLES

### Example 1

### Cytotoxic Activity of NK-92 Cells Against Polyomavirus-Positive Merkel Cell Carcinoma Cell Lines

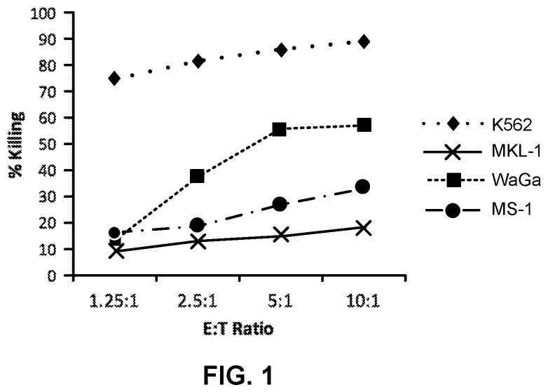

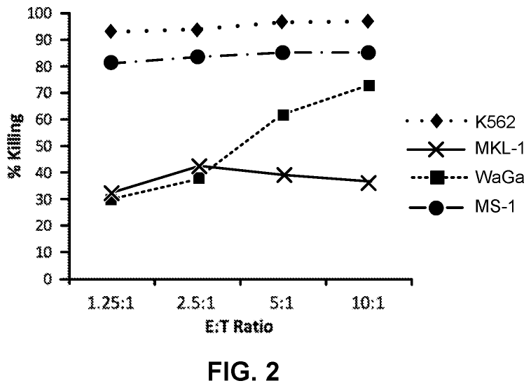

NK-92 cells demonstrate cytotoxic activity towards polyomavirus-positive MCC cell lines. FIGS. 1 and 2 show the results of NK-92 cell cytotoxicity after overnight exposure of NK-92 cells to three MCC cell lines (MKL-1, WaGa and MS-1) at different effector to target ratios. K562, a human CML cell line serves as a control, as it is consistently killed by NK-92 cells. Specifically, K562, MKL-1, MS-1, and WaGa cells (targets) were pre-stained with the membrane dye PKH67-GL, according to the manufacturer's instructions (Sigma Aldrich, St. Louis, Mo.), and resuspended in RPMI 1640+10% FBS at a cell density of 10e5/ml. NK-92 cells (effectors) were resuspended in X-Vivo10+5% HS+IL-2 (500 IU/ml) at a cell density of 10e6/ml. Target and effector cells were mixed in a 96-well plate at effector to target (E:T) ratios of 10:1, 5:1, 2.5:1, 1.25:1 in final volume of 200 ul/well. Targets alone controls were included to determine the spontaneous death background. The plate was incubated in a 37° C. CO₂ incubator for 4 hours or 24 hours, after which time the cells were stained with propidium iodide (0.1 μg/ml) for 10 minutes. Samples were analyzed by flow cytometry and the percentage cytotoxicity was calculated as follows: % Killing=[(% PKH+/PI+ in sample)−(% PKH+/PI+ in target alone)]/[100−(% PKH+/PI+ in target alone)]*100. FIG. 1 shows cytotoxicity at 4 hours and FIG. 2 shows cytotoxicity at 24 hours.

### Example 2

### Treatment of Merkel Cell Carcinoma (MCC) In Vivo Using NK-92 Cells

An 81 year old male patient with recurrent progressive MCC on the scalp with at least three cutaneous metastases was treated with NK-92 cells. Prior therapies had included surgery, adjuvant radiation (RT), intralesional interferon (IFN) plus RT plus topical imiquimod, anti-PD-1 therapy, intralesional TLR-4 agonist, RT with neutrons and octreotide-long-acting release (LAR). Patient received, in the first cycle on day 1, an NK-92 intravenous infusion of 2×10⁹ cells/m². On day 2 of the first cycle, patient received a second NK-92 infusion of 2×10⁹ cells/m². The cycle was repeated eight times with two week intervals between each cycle. The patient achieved a complete response (CR) with full resolution of the MCC tumors. The NK-92 therapy was tolerated with no significant adverse events.

A 75 year old male with progressive MCC on the thigh was treated with NK-92 cells. Prior therapies had included chemotherapy and anti-PD-1 therapy. Patient received, in the first cycle on day 1, an NK-92 intravenous infusion of 2×10⁹ cells/m². On day 2 of the first cycle, patient received a second NK-92 infusion of 2×10⁹ cells/m². The cycle was repeated a second time; however, therapy was discontinued due to a lack of significant change in disease state.

### Example 3

### Treatment of MCC Patients

Three patients were treated with NK-92 cells. These patients all had unresectable stage III (IIIB) or distant metastatic (stage IV) MCC based on Response Evaluation Criteria in Solid Tumors (RECIST). NK-92 cells were given to patients via IV infusion at a dose of 2×10e9 cells/m² on two consecutive days (=1 cycle) every 2 weeks for a total of 8 cycles (16 infusions). Patients were monitored and Progression Free Survival was assessed at 4 months from initiation of the treatment. The preliminary data showed that the NK-92 cell therapy achieved beneficial clinical results.

1. A method of treating merkel cell carcinoma in a subject, the method comprising:
(a) selecting a subject having merkel cell carcinoma;
(b) administering to the subject a therapeutically effective amount of NK-92 cells, wherein administration treats the merkel cell carcinoma in the subject.

2. The method of claim 1, wherein the subject has previously received radiation therapy, surgery, chemotherapy, anti-PD-1 therapy or any combination thereof.

3. The method of claim 1, wherein the merkel cell carcinoma is metastatic.

4. The method of claim 1, wherein from 1×10³ to 1×10¹⁰, per m² of the NK-92 cells are administered to the subject.

5. The method of claim 1, wherein 2×10⁹ per m², of the NK-92 cells are administered to the subject.

6. The method of claim 1, wherein the NK-92 cells are administered parenterally.

7. The method of claim 1, wherein the NK-92 cells are administered intravenously.

8. The method of claim 1, wherein the NK-92 cells are administered peritumorally.

9. The method of claim 1, wherein the NK-92 cells are administered to the subject by infusion over a period of time.

10. The method of claim 9, wherein the period of time is between 5 and 130 minutes.

11. The method of claim 9, wherein the period of time is between 90 and 120 minutes.

12. The method of claim 9, wherein the period of time is between 15 to 30 minutes.

13. The method of claim 1, wherein the merkel cell carcinoma is caused by the merkel cell polyomavirus.

14. The method of claim 1, wherein the merkel cell carcinoma is not caused by the merkel cell polyomavirus.

15. The method of claim 1, wherein the merkel cell carcinoma in the subject is resistant to chemotherapy.

16. The method of claim 1, wherein the NK-92 cells are administered to the subject once daily for 1, 2, 3, 4, 5, 6, 7, 8, 9, 10, 11, 12, 13, 14, 15, 16, 17, 18, 19, 20 or more days.

17. The method of claim 1, wherein the NK-92 cells are administered in a cycle of once daily for two days.

18. The method of claim 17, wherein the NK-92 cells are administered in 1, 2, 3, 4, 5, 6, 7, 8, 9, 10, 11, 12, 13, 14, 15, 16, 17, 18, 19, 20 or more cycles.
